(19) United States
(12) Patent Application Publication
(10) Pub. No.: US 2012/0116462 A1
(43) Pub. Date: May 10, 2012

(54) POLYAXIAL BONE SCREW

(75) Inventor: Jared ARAMBULA, San Diego, CA (US)

(73) Assignee: Alphatec Spine, Inc., Carlsbad, CA (US)

(21) Appl. No.: 13/288,225

(22) Filed: Nov. 3, 2011

Related U.S. Application Data
(60) Provisional application No. 61/411,557, filed on Nov. 9, 2010.

(51) Int. Cl. A61B 17/86

(52) U.S. Cl. 606/305

(57) ABSTRACT

A polyaxial bone screw includes a shaft, a body member, and a toroidal member. The shaft includes a longitudinal axis and a proximal end with a head having a generally rounded surface. The body member has a bore disposed longitudinally therethrough and a groove defined by a luminal surface of the bore. The toroidal member has an elliptical cross-section with a major axis and disposed within the groove to engage the generally rounded surface of the head at a point of contact.

### CROSS-REFERENCE TO RELATED APPLICATIONS

This application claims priority to U.S. Provisional Patent Application No. 61/411,557, filed Nov. 9, 2010, which is incorporated herein by reference.

### FIELD

The present invention relates generally to an apparatus for internal fixation of the spine and, more specifically relates to a polyaxial screw that is assembled in situ.

### BACKGROUND

Certain spinal conditions, including a fracture of a vertebra and a herniated disc, indicate treatment by spinal immobilization. Several systems of spinal joint immobilization are known, including surgical fusion and the attachment of pins and bone plates to the affected vertebras. Known systems include screws having proximal heads and threaded shafts that may be inserted into at least two spaced-apart vertebras. Each screw includes a body member attached over the head such that a stabilization rod can interconnect two or more body members to immobilize the vertebras spanned by the screws. However, in these systems, the body member is attached over the threaded shaft before the shaft is inserted into a vertebra.

During surgical implantation of spinal immobilization systems, the surgical site is crowded with tissue masses, sponges, and other surgical implements that may obstruct access to the sites of implantation of the threaded shafts. Further, because the body members are necessarily larger than the heads of the screws, it can be exceedingly difficult to implant a series of closely positioned screws. Current spinal immobilization systems would therefore benefit from a polyaxial screw including a body member that can be attached over a generally rounded proximal head of a threaded shaft subsequent to the implantation of the threaded shaft.

Thus, the present invention helps to alleviate a lack of space at the site of implantation of a spinal immobilization system as compared to the prior art, allowing the surgeon additional freedom in locating the threaded shafts of polyaxial bone screws closer together than previously possible. The result is a significantly improved polyaxial bone screw.

### SUMMARY

In one aspect of the present invention, a polyaxial bone screw includes a shaft, a body member, and a toroidal member. The shaft includes a longitudinal axis and a proximal end with a head having a generally rounded surface. The body member has a bore disposed longitudinally therethrough and a groove defined by a luminal surface of the bore. The toroidal member has an elliptical cross-section with a major axis and disposed within the groove to engage the generally rounded surface of the head at a point of contact.

In other features, the point of contact is distal to a point of intersection between the major axis and the head in a first position. The point of contact is proximal to a point of intersection between the major axis and the head in a second position. The toroidal member moves between a first position and a second position as the body member distally advances over the head. The body member removably attaches to the head by elastic deformation of the toroidal member. The body member removably attaches to the head by rotation of the toroidal member. The toroidal member comprises a coil spring. The toroidal member comprises a coil spring filled with an elastomer. The toroidal member comprises a toroidal sealing ring. The groove comprises a generally right angled internal shoulder.

In other features, the polyaxial bone screw includes a bushing with a pair of oppositely disposed proximally opening slots and distally depending tapered skirt adapted to abut a periphery of the head. The shaft includes a proximal portion with a circumference greater than a circumference the head to prevent proximal movement along the longitudinal axis by contact with the toroidal member.

In another aspect of the present invention a method for attachment of a body member of a polyaxial bone screw onto a head of the polyaxial bone screw includes the step of aligning a body member having a bore disposed longitudinally therethrough and a groove defined by a luminal surface of the bore relative to a shaft including a longitudinal axis and a proximal end with a head having a generally rounded surface. The method includes the step of advancing the body member over the head until a toroidal member having an elliptical cross-section with a major axis and disposed within the groove engages the generally rounded surface of the head at a point of contact, wherein the point of contact is distal to a point of intersection between the major axis and the head in a first position. The method includes the step of continuing to advance the body member over the head until the point of contact is proximal to the point of intersection between the major axis and the head in a second position.

In other features, the method further includes the step of continuing to advance body member moves the toroidal member between the first position and the second position as the body member distally advances over the head. The method further includes the step of elastically deforming the toroidal member as the body member advances relative to the head. The method further includes the step of rotating the toroidal member as the body member advances relative to the head. The method further includes the step of inserting a bushing with a pair of oppositely disposed proximally opening slots and distally depending tapered skirt adapted to abut a periphery of the head.

### BRIEF DESCRIPTION OF THE DRAWINGS

FIG. 1 is an elevational view of an embodiment of a polyaxial bone screw.

FIG. 2 is another elevational view of the polyaxial bone screw of FIG. 1.

FIG. 3 is a cross-sectional view of the polyaxial bone screw of FIG. 2 taken generally along the lines A-A of FIG. 2.

FIG. 4 is an enlarged view of a region represented by circular arrow B of FIG. 3.

FIG. 5 is a further enlarged view of FIG. 4, illustrating a body member in a state of removable attachment with a head of a threaded shaft.

FIG. 6 is a further enlarged view of FIG. 4, illustrating a body member in a state of permanent attachment with a head of a threaded shaft.

The foregoing and other features and advantages of the invention are apparent from the following detailed description of exemplary embodiments, read in conjunction with the accompanying drawings; wherein like structural or functional elements may be designated by like reference numerals.

### DETAILED DESCRIPTION

The words proximal and distal are applied to denote specific ends of components of the current invention described herein. A proximal end refers to the end of a component nearer to a medical professional when the component is implanted in a patient. A distal end refers to the end of a component further from the medical professional when the component is implanted in a patient.

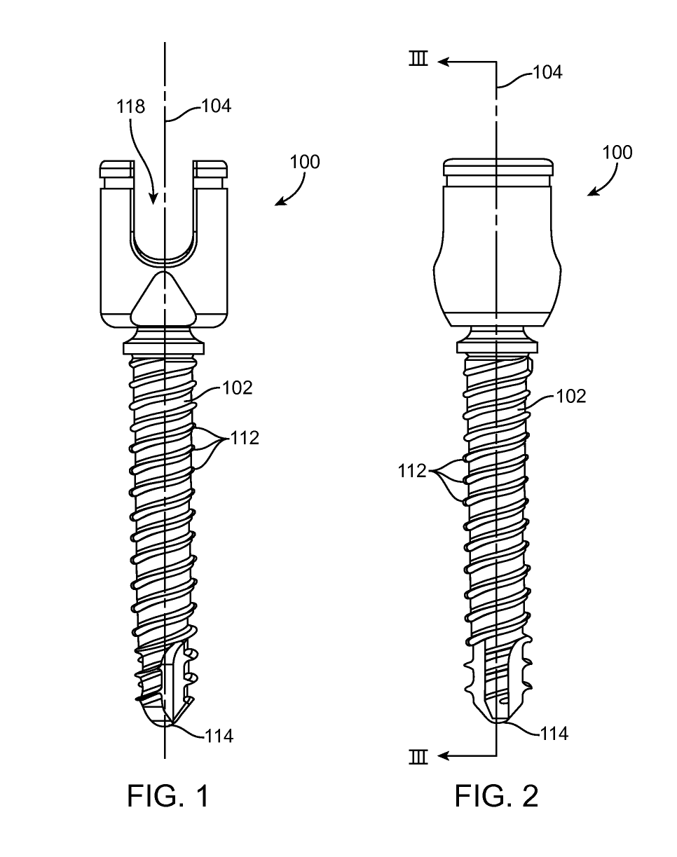

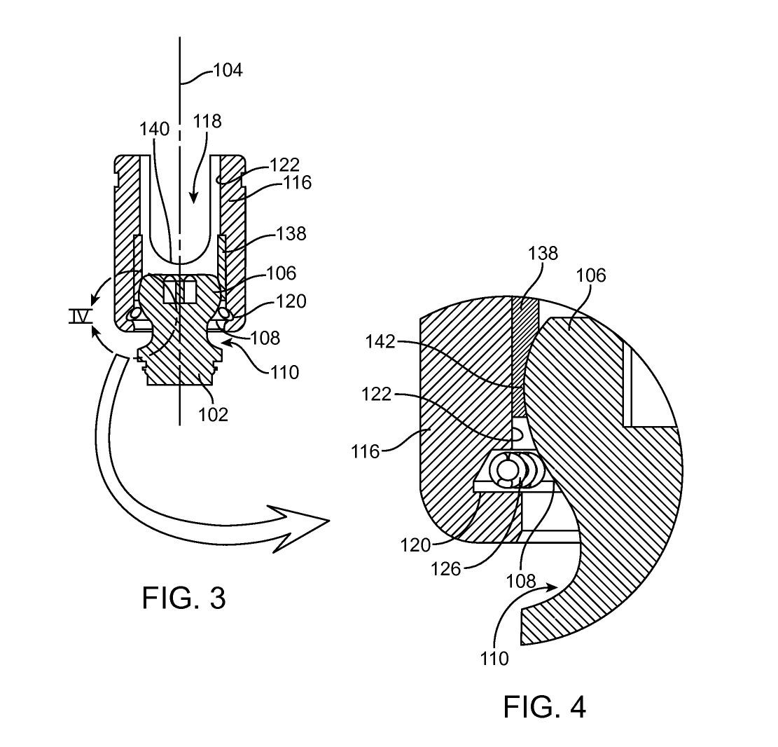

An embodiment of a polyaxial bone screw 100 includes a shaft 102 having a longitudinal axis 104, as illustrated in FIGS. 1 and 2. A head 106 having a generally rounded surface 108 is disposed at a proximal end 110 of the shaft 102, as illustrated in FIGS. 3-6. The shaft 102 may include threads 112 and a distal end 114 that is narrowed or pointed to facilitate entry into bony matter, as may be known in the art. Examples of shafts 102 that may be useful in the current invention may be found in Purcell et al. U.S. Patent Application Publication No. 2008/0243189, which is hereby incorporated by reference in its entirety herein. The generally rounded surface 108 provides variable angular movement between the head 106 and a body member 116 of the polyaxial bone screw 100.

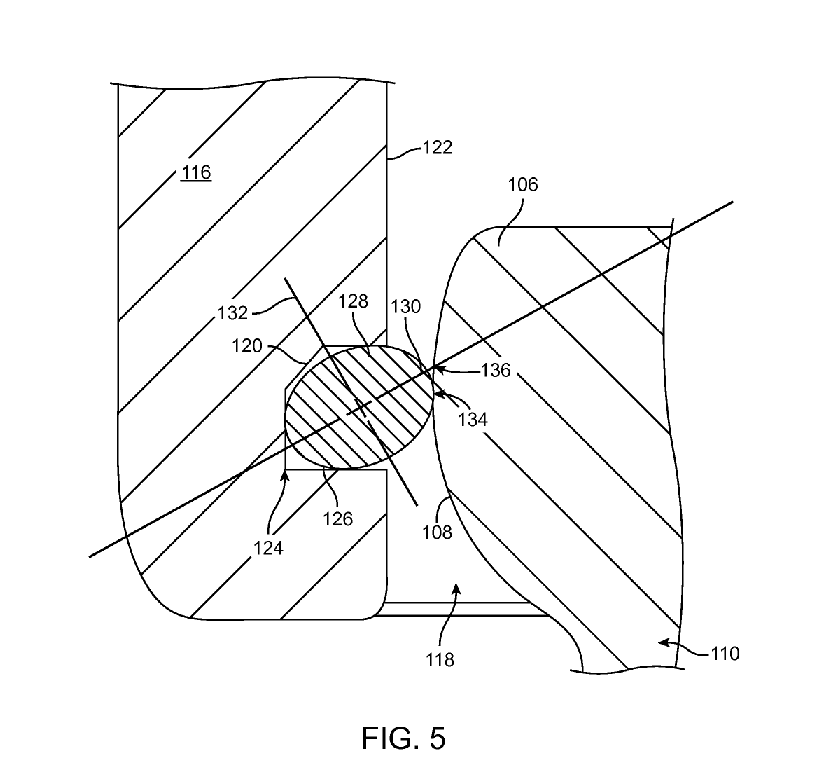

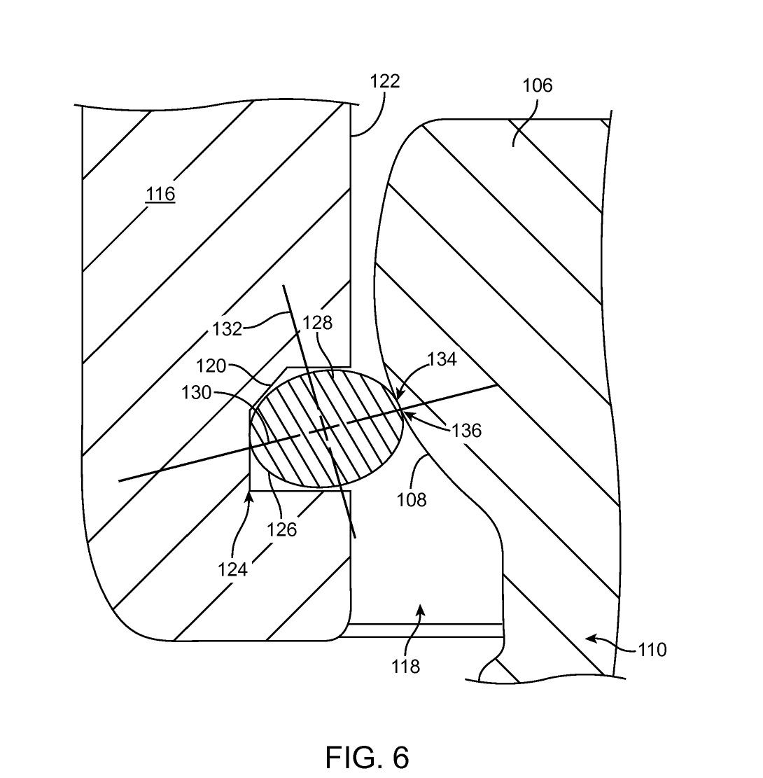

Referring to FIG. 3, the body member 116 includes a bore 118 disposed longitudinally therethrough. In one embodiment, the bore 118 includes a circumference or perimeter greater than the circumference or perimeter of the head 106, such that the head 106 may be coaxially disposed within the bore 118. A groove 120 is defined by a luminal surface 122 of the bore 118 and is disposed on the distal portion of the body member 116. As illustrated in FIGS. 4-6, the groove 120 includes a generally right angled internal shoulder 124 therein; alternatively, obtuse or acute angles may be used for the internal shoulder 124 as to accommodate a toroidal member 126. The toroidal member 126 includes an elliptical cross-section 128 and is disposed within the groove 120. The toroidal member 126 may comprise, for example, a toroidal spring coil, a toroidal sealing ring, a toroidal spring coil filled with an elastomer, or other flexible toroidal structures including an elliptical cross-section. The elliptical cross-section 128 includes a major axis 130 and a minor axis 132, as illustrated in FIGS. 5 and 6.

The body member 116 circumferentially attaches over the head 106 subsequent to advancement of the shaft 102 into a bone. Subsequent attachment of the body member 116 allows a medical professional to implant adjacently placed shafts 102 closer together than would be possible if the body member 116 were attached to the shaft 102 prior to implantation thereof. In one embodiment of a method for attachment of the body member 116 to the head 106, the body member 116 is coaxially aligned with the head 106 such that the bore 118 is positioned to accommodate the head 106. Such coaxial alignment is illustrated by the cross-sectional view of FIG. 3. Attachment is achieved by forcing the distal portion of the body member 116 toward the head 106 such that the body member 116 attaches to the head 106 via the toroidal member 126, as illustrated in FIGS. 3-6. Examples of attachment mechanisms that may be useful in the current invention and that utilize a toroidal member 126 having an elliptical cross-section 128, where the toroidal member 126 is disposed within a groove 120 including a right-angle internal shoulder 124, may be found in Balsells U.S. Pat. No. 5,134,244 and Balsells U.S. Pat. No. 7,210,398, both of which are hereby incorporated in their entirety by reference herein. In one embodiment, the proximal portion 110 of the shaft 102 includes a circumference or perimeter that is greater than the circumference or perimeter of the head 106, thus, when the body member 116 attaches to the head 106 through the toroidal member 126, any proximal movement along the longitudinal axis 104 of the shaft 102 is prevented by the toroidal member 126.

Referring to FIGS. 5 and 6, the body member 116 is movably attached to the head 106; thus, the body member 116 may be rotated about the longitudinal axis 104. Alternatively or in addition, the body member 116 may be disposed such that a longitudinal axis of the body member 116 is not parallel to the longitudinal axis 104 of the shaft 102. Such non-parallel disposition facilitates the polyaxial nature of the polyaxial bone screw 100, for example, the shaft 102 may be disposed at an angle relative to the body member 116 by way of the rounded surface 108 of the head 106.

Referring to FIG. 5, removable attachment of the body member 116 to the head 106 may be achieved by forcing the body member 116 distally over the head 106 at least until a point of contact 134 between the toroidal member 126 and the head 106 is distal to an intersection 136 of the major axis 130 of the elliptical cross-section 128 and the head 106. In the current context, “removable” means that the body member 116 may be disengaged from the head 106 without damage to the toroidal member 126 or the body member 116. Referring to FIG. 6, permanent attachment of the body member 116 to the head 106 may be achieved by forcing the body member 116 distally over the head 106 at least until the point of contact 134 is proximal to the intersection 136 of the major axis 130 of the elliptical cross-section 128 and the head 106.

Embodiments of the polyaxial bone screw 100 described hereinabove may be tailored to include predetermined attachment characteristics. For example, the size and shape of the groove 120, as well as the toroidal member 126 may be adjusted to achieve removable or permanent attachment of the body member 116 to the head 106 at a desired point of attachment. Another factor in the determination of the desired point of attachment may be the distance between the luminal surface 122 and a closest point thereto on the generally rounded surface 108. A further factor may be the curvature of the generally rounded surface 108.

It is contemplated that another embodiment of the polyaxial bone screw 100 includes a pressure bushing 138 disposed on the luminal surface 122 of the body member 116, as illustrated in FIGS. 3 and 4. In one embodiment, the pressure bushing 138 includes a pair of oppositely disposed proximally opening slots 140 and a distally depending tapered skirt 142. The slots 140 accommodate a fixation rod (not shown) and the distally depending tapered skirt 142 accommodates a periphery of the head 106. Examples of body members 116 and pressure bushings 118 that may be useful in the current invention may be found in Purcell et al. U.S. Patent Application Publication No. 2008/0243189.

An improved polyaxial bone screw for a spinal immobilization system is presented. The polyaxial bone screw includes a body member attachable to a head of a shaft subsequent to advancement of the shaft into bone. Such subsequent attachment facilitates implantation of the shafts closer together than would otherwise be practical due to the presence of a body member attached to each shaft before such implantation.

It will be appreciated by those skilled in the art that changes could be made to the embodiments described hereinabove without departing from the broad concepts disclosed therein. It is understood, therefore, that this disclosure is not limited to the particular embodiments disclosed, but it is intended to cover modifications that may include a combination of features illustrated in one or more embodiments with features illustrated in any other embodiments. Various modifications, equivalent processes, as well as numerous structures to which the present disclosure may be applicable will be readily apparent to those of skill in the art to which the present disclosure is directed upon review of the present specification. Accordingly, this description is to be construed as illustrative only and is presented for the purpose of enabling those skilled in the art to make and use the polyaxial bone screw described herein and to teach the best mode of carrying out the same.

The invention claimed is:

1. A polyaxial bone screw, comprising:
a shaft including a longitudinal axis and a proximal end with a head having a generally rounded surface;
a body member having a bore disposed longitudinally therethrough and a groove defined by a luminal surface of the bore; and
a toroidal member having an elliptical cross-section with a major axis and disposed within the groove to engage the generally rounded surface of the head at a point of contact.

2. The polyaxial bone screw of claim 1, wherein the point of contact is distal to a point of intersection between the major axis and the head in a first position.

3. The polyaxial bone screw of claim 1, wherein the point of contact is proximal to a point of intersection between the major axis and the head in a second position.

4. The polyaxial bone screw of claim 1, wherein the toroidal member moves between a first position and a second position as the body member distally advances over the head.

5. The polyaxial bone screw of claim 1, wherein the body member removably attaches to the head by elastic deformation of the toroidal member.

6. The polyaxial bone screw of claim 1, wherein the body member removably attaches to the head by rotation of the toroidal member.

7. The polyaxial bone screw of claim 1, wherein the toroidal member comprises a coil spring.

8. The polyaxial bone screw of claim 1, wherein the toroidal member comprises a coil spring filled with an elastomer.

9. The polyaxial bone screw of claim 1, wherein the toroidal member comprises a toroidal sealing ring.

10. The polyaxial bone screw of claim 1, wherein the groove comprises a generally right angled internal shoulder.

11. The polyaxial bone screw of claim 1, further comprising a bushing with a pair of oppositely disposed proximally opening slots and distally depending tapered skirt adapted to abut a periphery of the head.

12. The polyaxial bone screw of claim 1, wherein the shaft includes a proximal portion with a circumference greater than a circumference the head to prevent proximal movement along the longitudinal axis by contact with the toroidal member.

13. A method for attachment of a body member of a polyaxial bone screw onto a head of the polyaxial bone screw, comprising:
aligning a body member having a bore disposed longitudinally therethrough and a groove defined by a luminal surface of the bore relative to a shaft including a longitudinal axis and a proximal end with a head having a generally rounded surface;
advancing the body member over the head until a toroidal member having an elliptical cross-section with a major axis and disposed within the groove engages the generally rounded surface of the head at a point of contact, wherein the point of contact is distal to a point of intersection between the major axis and the head in a first position; and
continuing to advance the body member over the head until the point of contact is proximal to the point of intersection between the major axis and the head in a second position.

14. The method of 13, wherein continuing to advance body member moves the toroidal member between the first position and the second position as the body member distally advances over the head.

15. The method of claim 13, further comprising elastically deforming the toroidal member as the body member advances relative to the head.

16. The method of claim 13, further comprising rotating the toroidal member as the body member advances relative to the head.

17. The method of claim 13, further comprising inserting a bushing with a pair of oppositely disposed proximally opening slots and distally depending tapered skirt adapted to abut a periphery of the head.
